(19) United States
(12) Patent Application Publication
(10) Pub. No.: US 2004/0228890 A1
(43) Pub. Date: Nov. 18, 2004

(54) Two-coat cosmetic product, its uses, and makeup kit including the product

(76) Inventors: Xavier Blin, Paris (FR); Caroline Lebre, Thiais (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/784,949

(22) Filed: Feb. 25, 2004

Related U.S. Application Data
(60) Provisional application No. 60/452,109, filed on Mar. 6, 2003.

(30) Foreign Application Priority Data
Feb. 25, 2003 (FR) 03 02309

(51) Int. Cl. A61K 7/00

(52) U.S. Cl. 424/401

(57) ABSTRACT

Disclosed herein is a cosmetic product comprising at least two compositions wherein the first composition comprises a liquid fatty phase and polymer particles dispersed in the liquid fatty phase and the second composition comprises a high molecular weight polymer having a weight-average molecular mass of greater than or equal to 200 000 g/mol; also disclosed are a makeup method and a makeup kit including the product, which may be in the form of a lipstick.

This application claims benefit of U.S. Provisional Application No. 60/452,109, filed Mar. 6, 2003.

The present disclosure relates to a new cosmetic product comprising at least two compositions that can be applied, in succession, to, for example, the skin of both the face and the human body, to the upper and lower eyelids of human beings, to the lips and to the epidermal derivatives, such as the nails, eyebrows, eyelashes or hair. This disclosure also relates to an at least two-coat makeup method for the face and human body.

One of the at least two compositions may be chosen from a loose powder, compact powder, a foundation, a blusher, eyeshadow, concealer, lipstick, lip balm, lipgloss, lip pencil, eye pencil, mascara, eyeliner, nail varnish, a body makeup, and skin coloring product.

This new cosmetic product provides, for instance, a glossy, taste-free makeup that has good coverage and staying power and that does not transfer.

This disclosure also relates to, for example, a composition for caring for and/or making up keratin materials such as the skin of both the face and the human body, including the scalp, the epidermal derivatives, such as the eyelashes, eyebrows, nails and hair, and also the lips and upper or lower eyelids of human beings.

“Non-transfer” makeup compositions for the lips and skin may have the advantage of forming a deposit, which, at least in part, is not deposited on the substrates with which they may be brought into contact, for instance, glasses, clothes, and cigarettes.

Some known non-transfer compositions, such as those disclosed in U.S. Pat. No. 6,074,654 and WO 02/067877, are generally based on silicone resins and volatile silicone oils and, although they may have improved staying properties, they may also have the drawback of leaving on the skin and lips, following evaporation of the volatile silicone oils, a film which over time becomes uncomfortable, for example, giving sensations of drying and tightening, thereby causing a certain number of women not to use this type of product.

To remedy some of these disadvantages, the present inventors experimented with the production of makeup compositions comprising particles of polymer dispersed and surface-stabilized by a stabilizer in a liquid fatty phase, as described, for example, in EP-A-0 930 060. When, however, these compositions did not provide the kind of truly glossy makeup always sought after by consumers, the inventors further experimented with the application to this composition of a topcoat comprising a mixture of a silicone oil with a viscosity of 20 cSt and a silicone oil with a viscosity of 10 000 cSt, for example, see patent application EP 1 249 223. That composition, however, has the disadvantage of migrating and of becoming oily.

The company Kose, moreover, in its patent application JP-A-0 5 221 829, proposed the use of a gel based on perfluorinated substances, which is applied over a lipstick film so as to prevent its transfer to other surfaces, the gel being incompatible with the lipstick film. Although the use of perfluorinated oils may ensure incompatibility between the gel and the lipstick film, and hence properties of staying power and absence of transfer, the formulations of this type have the disadvantage of poor cosmetic properties, such as, the lipstick film may become oily and liable to migrate, which is unacceptable to consumers.

In patent application WO-A-97/17057, the company Procter & Gamble describes a method of enhancing the staying and non-transfer properties, which consists in applying two compositions one over the other. These two compositions meet the following physicochemical criteria:

overall Hildebrand solubility parameters less than 8.5 (cal/cm³)^{1/2}, for the composition applied first;

the presence of oil whose calculated partition coefficient ClogP is at least 13, for the topcoat.

However, the compositions, given by way of example, provide an uncomfortable makeup composition.

Application WO 02/067877 describes a method of improving the aesthetic properties of a non-transfer composition, which consists in applying a second composition to a film of non-transfer composition. Some of the products described in this document have an unpleasant odor and are sticky. Other products lack sufficient gloss and transparency.

Finally, U.S. Pat. No. 6,001,374 to Nichols proposes a multi-layer makeup system which involves using a base composition, containing a resin which is soluble in alcohol and insoluble in water, which can be applied as a base coat, and which may not stain any of the substrates brought into contact with the makeup, and may resist water and rubbing, while having a certain gloss. This composition, however, contains a water-soluble alcohol, in particular ethanol, a compound which has an irritant character, is drying to the skin and more specifically to the lips, and which is excessively uncomfortable.

U.S. Patent No. 6,001,374 to Nichols likewise proposes a topcoat composition which contains a silicone compound. The silicone compounds cited as examples, particularly the polydimethylsiloxane with a viscosity of 20,000,000 cSt and a molecular weight of 500,000, in solution in a polydimethylsiloxane with a viscosity of 5 cSt and a molecular weight of 800, in proportions by mass of 15/85 (reference product SF 1236), are excessively sticky.

One aspect of the present invention is to provide a makeup product that exhibits the properties of “non-transfer”, non-migration, staying power, comfort, absence of drying, and gloss, a result that has not heretofore been satisfactorily obtained.

The present inventors have found, surprisingly, that combining a first composition comprising a liquid fatty phase and polymer particles dispersed in the fatty phase and a second composition comprising a high molecular weight polymer having a weight-average molecular mass of greater than or equal to 200,000 g/mol, may give a glossy two-coat makeup which has no taste, does not migrate and does not transfer, while being comfortable on application and overtime, e.g., no drying, no tightening.

For example, the product of the present disclosure makes it possible to obtain continuous deposits that do not have an unpleasant taste, are not sticky, provide good coverage, have a glossy appearance, do not migrate or undergo transfer, stay on well, are not oily and/or do not dry out the skin or lips to which the product is applied, either at the time of application or over time. In addition, it has good stability properties and hence allows a homogeneous and aesthetic makeup.

It has also been found that the compositions used in the method according to the present disclosure have the sought-after qualities of, for example, spreading on and adhering to the skin, the lips, the eyelashes or the mucous membranes, and also exhibit a creamy and pleasant feel.

These properties of staying power and of absence of taste, transfer and migration, allied to the non-greasy, comfortable and glossy appearance, make this a product that is suitable for, for instance, producing lip makeup products such as lipsticks and lipglosses, or eye makeup products such as mascara, eyeliners and eyeshadows.

A “liquid fatty phase,” as the term is used in the present disclosure, is any non-aqueous medium that is liquid at ambient temperature (25° C.) and atmospheric pressure (760 mm Hg), which is composed of at least one fatty substance that is liquid at ambient temperature, for example, oil, with the exception of the plasticizer, and also gelling agents and pigment stabilizers that may be present in the composition, provided that they are liquid at ambient temperature and atmospheric pressure. The liquid fatty phase may comprise a volatile liquid fatty phase and/or a non-volatile fatty phase.

As disclosed herein, a “non-volatile fatty phase” is any medium apt to remain on the skin or lips without evaporating. A non-volatile fatty phase has, for example, a non-zero vapor pressure at ambient temperature and atmospheric pressure, of less than 0.02 mm Hg and, for instance, less than 10⁻³ mm Hg.

As disclosed herein, a “volatile fatty phase” is any non-aqueous medium apt to evaporate from the skin or lips at ambient temperature and atmospheric pressure. This volatile phase comprises, for example, oils having a vapor pressure at ambient temperature (25° C.) and atmospheric pressure (760 mm Hg) ranging from 10⁻³ to 300 mm Hg (0.13 Pa to 40 000 Pa), such as from 0.2 to 300 mm Hg (2.66 Pa to 40 000 Pa).

The term “hydrocarbon oil” refers to oils comprising primarily carbon atoms and hydrogen atoms, and for instance, alkyl or alkenyl chains, such as alkanes or alkenes, and also to oils having an alkyl or alkenyl chain comprising at least one group chosen from ether, ester and carboxylic acid groups.

As used in the present disclosure, the term “substituted” means further comprising at least one substituent chosen from an oxygen, a nitrogen, a halogen, hydroxyl, ether, oxyalkylene, polyoxyalkylene, carboxyl, amine and amide groups.

The term “saccharide” refers to a monosaccharide or a polysaccharide.

“Polar groups” are known to the persons of ordinary skill in the art. For example, polar groups may be, ionic and/or non-ionic polar groups chosen from —COOH; —OH; ethylene oxide; propylene oxide; —PO₄; —NHR; —NR₁R₂, wherein R₁ and R₂, which may be identical or different, may form a ring and are chosen from linear and branched C₁ to C₂₀ alkyl and alkoxy radicals.

A “gelling agent” is any compound that increases the viscosity of the medium into which it is incorporated, or which stiffens the medium. The gelling agent according to the present disclosure does not include waxes.

A “wax,” for the purposes of the present disclosure, is a lipophilic fatty compound that is solid at ambient temperature (25° C.) and exhibits a reversible solid/liquid state change, the compound having a melting temperature of greater than 40° C. and possibly up to 200° C. and having an anisotropic crystalline organization in the solid state. The size of the crystals is such that they diffract and/or scatter light, thereby endowing the composition with a turbid, more or less opaque appearance. By bringing the wax to its melting temperature it can be made miscible with the oils and made to form a microscopically homogeneous mixture; however, by bringing the temperature of the mixture back to ambient temperature, the wax is recrystallized in the oils of the mixture. It is this recrystallization in the mixture which is responsible for the decrease in the gloss of the mixture.

An “ungrafted polymer” is a polymer obtained by polymerizing at least one monomer without subsequent reaction of the side chains with another chemical compound.

A “liquid silicone compound” is a silicone compound which is liquid at ambient temperature (25° C.) and atmospheric pressure (760 mm Hg).

Accordingly, disclosed herein is a cosmetic product, for example, a makeup product, comprising at least two compositions, wherein a first composition comprises a liquid fatty phase and polymer particles dispersed in the fatty phase and a second composition comprises a high molecular weight polymer having a weight-average molecular mass of greater than or equal to 200 000 g/mol.

A “makeup product” can be any product comprising a colorant that allows a color to be deposited on a human keratin material, for instance, the skin, lips or epidermal derivatives, by applying to the keratin material products such as lipsticks, rouges, eyeliners, foundations, self-tanning products, and semi-permanent makeup products (tattoos).

When the makeup product is intended for making up the lips, the first composition may be in the form of a stick or in fluid form, and the second composition may also be in the form of a stick or in fluid form. A lip makeup product as disclosed herein may comprise for example, a first composition in the form of a stick, it being possible for the second composition to be in the form of a stick, or in fluid form, packaged in a tube. The product according to the present disclosure comprises at least two physiologically acceptable compositions that can be packaged separately, or together in a single container, or in at least two separate or discrete containers.

According to one aspect of the present disclosure, the at least two compositions are packaged separately, and for example, in a single container having two ends.

The present disclosure accordingly provides, for example, a cosmetic makeup product in the form of a foundation, blusher, eyeshadow, lipstick, product having care properties for instance, eyeliner, concealer or body makeup product (of the tattoo type). For example, the cosmetic makeup product as disclosed herein may be a lipstick.

Further disclosed herein is a makeup kit comprising a cosmetic makeup product as defined above, in which the different compositions are packaged separately and are accompanied by appropriate means of application. Appropriate means of application may include fine or coarse brushes, pens, pencils, felts, quills, sponges, tubes and/or foam ends.

The first composition of the product according to the present disclosure may be applied to the keratin material to form a basecoat, over which the second composition is deposited to give a topcoat or finish coat. It is possible to apply under the basecoat an undercoat, whose composition may be the same as or different from that of the second coat.

It is also possible to deposit over the first topcoat a second topcoat, whose composition is the same as or different from that of the first topcoat. The resulting makeup is, for example, a makeup comprising a basecoat and a topcoat.

The first composition can be, for example, a foundation, a rouge, a lipstick, a lipgloss, an eyeliner or a body makeup product and the second composition can be, for example, a care product or a product intended for preserving and/or enhancing the cosmetic properties of the first composition.

The present disclosure additionally relates to a method of making up the skin and/or lips and/or epidermal derivatives, which comprises applying to the skin and/or lips and/or epidermal derivatives a cosmetic makeup product as defined above.

The present disclosure further provides a method of making up the skin and/or lips and/or epidermal derivatives of a human being, which comprises applying to the skin, lips and/or epidermal derivatives a first coat of a first composition comprising a fatty phase and polymer particles dispersed in the fatty phase and then applying over all or part of the first coat a second coat of a second composition comprising a high molecular weight polymer having a weight-average molecular mass of greater than or equal to 200 000 g/mol.

For example, the method according to the present disclosure may comprise applying to the skin, lips and/or epidermal derivatives of a human being a first coat of a first composition comprising a fatty phase and polymer particles dispersed in the fatty phase, leaving the said first coat to dry if necessary, and then applying over all or part of the first coat a second coat of a second composition comprising a high molecular weight polymer having a weight-average molecular mass of greater than or equal to 200,000 g/mol.

This two-coat makeup may be adapted to all products for making up the skin of both the face and the body, the mucosae, such as the lips, the interior of the lower eyelids, and epidermal derivatives, such as the nails, eyelashes, hair, eyebrows and even body hairs. The second coat may form designs and may be applied with a pen, pencil or any other instrument, for example, sponge, finger, fine or coarse brush, quill, etc. The makeup may also be applied to makeup accessories, such as false nails, false eyelashes, hairpieces or else discs or patches which adhere to the skin or lips, e.g., of the beauty-spot type.

The disclosure additionally provides a made-up substrate comprising a first coat of a first composition comprising a fatty phase and polymer particles dispersed in the fatty phase and a second coat of a second composition deposited over all or part of the first coat and comprising at least one high molecular weight polymer having a weight-average molecular mass of greater than or equal to 200 000 g/mol.

Finally, the present disclosure relates to a method for providing the skin and/or lips and/or epidermal derivatives with a makeup that exhibits at least one characteristic chosen from comfort, gloss, non-transfer properties, non-migration properties, and staying power, by making a cosmetic make up product having at least two compositions as described above and applying it to the skin and/or lips and/or epidermal derivatives.

First Composition

The first composition according to the present disclosure comprises a liquid fatty phase and polymer particles dispersed in the said liquid fatty phase.

Dispersed Polymer Particles

According to the present disclosure, the polymer of the first composition is a solid that is insoluble in the liquid organic phase of the first composition even at its softening temperature, in contradistinction to a wax, even a wax of polymeric origin, which is soluble in the liquid organic phase (or fatty phase) at its melting temperature. In addition it allows the formation of a deposit, for example, a film-forming deposit, which is continuous and homogeneous and/or it is characterized by the interpenetration of the polymeric chains. With a wax, even one obtained by polymerization, melting in the liquid organic phase is followed by recrystallization. It is believed that this recrystallization that is responsible, for instance, for the loss of gloss of the composition.

To optimize the non-transfer properties the amount of polymer is selected as a function of the amount of colorants and/or actives and/or oils present in the first composition. For example, the amount of polymer, in terms of active material, may be greater than 5% by weight, relative to the total weight of the composition.

One benefit of using a dispersion of polymer particles in a composition as disclosed herein, is that these particles remain in the state of elementary particles, without forming agglomerates, in the fatty phase. Another benefit of the polymer dispersion is the possibility of obtaining highly fluid compositions, such as of the order of 130 centipoises, even in the presence of a high proportion of polymer.

Yet another benefit of a polymer dispersion of this kind is that the polymer particle size can be calibrated at will and its size “polydispersity” modified during the synthesis. It is thereby possible to obtain very small-sized particles that are invisible to the naked eye when in the composition and when applied to the skin, lips or epidermal derivatives.

A further benefit of the polymer dispersion of the composition of the present disclosure is the possibility of varying the glass transition temperature (Tg) of the polymer or polymeric system, e.g., polymer and plasticizer-type additive and hence the possibility of changing from a hard polymer to a more or less soft polymer, thereby allowing the mechanical properties of the composition to be regulated as a function of the intended application, for instance, of the film deposited.

The first composition of the product as disclosed herein, therefore may comprise, for example, at least one stable dispersion of polymer particles, such as spherical particles of at least one polymer, in a physiologically acceptable liquid organic phase. These dispersions may, for example, be in the form of polymer nanoparticles in stable dispersion in the liquid organic phase. The nanoparticles can be, for example, of an average size ranging from 5 to 800 nm, for instance ranging from 50 to 500 nm. It is possible, however, to obtain polymer particle sizes ranging up to 1 μm.

The polymer particles in dispersion may be, for instance, insoluble in water-soluble alcohols such as ethanol, for example.

The polymers in dispersion that can be used in the first composition of the present disclosure may have, for example, a molecular weight ranging from 2,000 to 10,000,000 g/mol, and a T_{g} ranging from −100° C. to 300° C., such as from −50° C. to 100° C., for instance, from −10° C. to 50° C.

When the polymer has a glass transition temperature that is too high for the desired application it is possible to combine it with a plasticizer so as to lower the glass transition temperature of the mixture used. The plasticizer may be chosen from the plasticizers commonly used in the field of the application of the end product, for example, from compounds able to act as solvents for the polymer. It is additionally possible to use coalescence agents in order to promote the formation of a continuous and homogeneous deposit of the polymer.

Non-limiting examples of coalescence agents or plasticizers which can be used in the present disclosure are those referred to in FR-A-2 782 917.

It is possible to use filmable polymers, for example, those having a low T_{g}, such as less than or equal to the temperature of the skin, for instance, less than or equal to 40° C. As disclosed herein, a polymer that is “filmable” is able, alone or in combination with a plasticizer, to form an isolatable film.

It is also possible to use a “non-filmable polymer” herein. A “non-filmable polymer” is a polymer which is not capable on its own of forming an isolatable film. In combination with a non-volatile compound of the oil type, this polymer allows a continuous and homogeneous deposit to be formed on the skin and/or lips.

Among filmable polymers, non-limiting mention may be made of radical, acrylic or vinyl homopolymers or copolymers, for example, having a T_{g} of less than or equal to 40° C., such as ranging from −10° C. to 30° C., which are used alone or in a mixture.

Among non-filmable polymers, non-limiting mention may be made of radical, vinyl or acrylic homopolymers or copolymers, optionally crosslinked, having a T_{g} of, for example, greater than 40° C., such as ranging from 45° C. to 150° C., which may be used alone or in a mixture.

A “radical polymer” is a polymer obtained by polymerizing monomers containing unsaturation, such as ethylenic unsaturation, each monomer being capable of undergoing homopolymerization (in contrast to polycondensates). The radical polymers may be, for example, vinyl polymers or copolymers, such as acrylic polymers. The acrylic polymers may result from the polymerization of ethylenically unsaturated monomers having at least one acid group and/or esters of these acid monomers and/or amides of these acids.

As a monomer carrying an acid group it is possible to use α,β-ethylenic unsaturated carboxylic acids such as acrylic acid, methacrylic acid, crotonic acid, maleic acid and itaconic acid. For example, in one aspect of the present disclosure, the monomer carrying an acid group is chosen from (meth)acrylic acid and crotonic acid, such as (meth)acrylic acid.

The esters of acid monomers, for example, can be chosen from esters of (meth)acrylic acid (also called (meth)acrylates), such as alkyl (meth)acrylates, for instance, C₁-C₂₀, such as C₁-C₈, alkyl (meth)acrylates, aryl (meth)acrylates, for example C₆-C₁₀ aryl (meth)acrylates, and hydroxyalkyl (meth)acrylates, for instance, C₂-C₆ hydroxyalkyl (meth)acrylates. Alkyl (meth)acrylates include methyl, ethyl, butyl, isobutyl, 2-ethylhexyl and lauryl (meth)acrylate. Hydroxyalkyl (meth)acrylates include hydroxyethyl (meth)acrylate and 2-hydroxypropyl (meth)acrylate. Aryl (meth)acrylates include benzyl acrylate and phenyl acrylate.

Non-limiting mention may be made of esters of (meth)acrylic acid, such as alkyl (meth)acrylates.

As the radical polymer, examples that can be used include copolymers of (meth)acrylic acid and alkyl (meth)acrylate, such as C₁-C₄ alkyl (meth)acrylate. For instance, it is possible to use methyl acrylates optionally copolymerized with acrylic acid.

Amides of the acid monomers include (meth)acrylamides, such as N-alkyl (meth)acrylamides, for example N-C₂-C₁₂ alkyl(meth)acrylamides, such as N-ethylacrylamide, N-t-butylacrylamide and N-octylacrylamide, and N-di(C₁-C₄)alkyl(meth)acrylamides.

The acrylic polymers may likewise result from the polymerization of ethylenically unsaturated monomers having at least one amine group, in free form or else in partly or totally neutralized form, or else in partly or totally quaternized form. Such monomers may be, for example, dimethylaminoethyl (meth)acrylate, dimethylaminoethylmethacrylamide, vinylamine, vinylpyridine and diallydimethylammonium chloride.

The vinyl polymers may likewise result from the homopolymerization or copolymerization of at least one monomer chosen from vinyl esters and styrenic monomers. For example, these monomers may be polymerized with acid monomers and/or their esters and/or their amides, such as those mentioned above. Examples of vinyl esters include vinyl acetate, vinyl propionate, vinyl neodecanoate, vinyl pivalate, vinyl benzoate and vinyl t-butylbenzoate. Styrenic monomers include styrene and alpha-methylstyrene.

The list of monomers given is in no way limiting and it is possible to use any monomer known to the person skilled in the art which falls within the categories of acrylic monomers and vinyl monomers, including monomers modified with a silicone chain.

Of other vinyl monomers that can be used, non-limiting mention may also be made of the following:

N-vinylpyrrolidone, vinylcaprolactam, vinyl-N-(C₁-C₆)alkylpyrroles, vinyloxazoles, vinylthiazoles, vinylpyrimidines and vinylimidazoles; and

olefins such as ethylene, propylene, butylene, isoprene and butadiene.

The vinyl polymer may be crosslinked using at least one difunctional monomers, including for example, at least two ethylenic unsaturations, such as ethylene glycol dimethacrylate and diallyl phthalate.

Without limitation, the polymers in dispersion of the present disclosure may be chosen from the following polymers and copolymers: polyurethanes, acrylic polyurethanes, polyureas, polyurea-polyurethanes, polyester-polyurethanes, polyether-polyurethanes, polyesters, polyester amides, fatty-chain polyesters, alkyds; acrylic and/or vinyl polymers or copolymers; acrylic-silicone copolymers; polyacrylamides; silicone polymers such as silicone polyurethanes and silicone acrylics, fluoro polymers, and mixtures thereof.

The at least one polymer in dispersion in the organic liquid phase may be present in the composition in an amount ranging from 2 to 40% by weight, relative to the dry-matter content of the composition, such as from 5 to 40%, for instance from 5 to 35%, and for example, from 8 to 30%.

In one aspect of the present disclosure, the polymer particles in dispersion are surface-stabilized by a stabilizer that is solid at ambient temperature. In that case, the amount of the dispersion in terms of dry matter is equal to the total amount of polymer+stabilizer, provided that the amount of polymer may not be less than 5%.

In another aspect of the present disclosure, a dispersion of filmable polymer is used, with the particles being dispersed in a volatile oil.

Polymer Particle Stabilizer

The polymer particles used in the first composition of the present disclosure are for instance, surface-stabilized by means of a stabilizer which may be chosen from block polymers, graft polymers andrandom polymers, either alone or as a mixture. Stabilization can be effected by any known means, for example, by direct addition of the block polymer, graft polymer and/or random polymer during the polymerization.

The stabilizer, for example, may also be present in the mixture before polymerization of the polymer. However, it is also possible to add the stabilizer continuously, such as when the monomers are also added continuously.

The stabilizer may be present in the composition in an amount ranging from 2-30% by weight of stabilizer, relative to the initial mixture of monomers, such as from 5-20% by weight.

When a graft polymer and/or block polymer is used as stabilizer, the synthesis solvent is chosen such that at least some of the grafts or blocks in the stabilizing polymer are soluble in the solvent, the remainder of the grafts or blocks not being soluble therein. The stabilizing polymer used during the polymerization should be soluble or dispersible in the synthesis solvent. Furthermore, for example, a stabilizer can be chosen whose insoluble blocks or grafts have a certain affinity for the polymer formed during the polymerization.

Among graft polymers that may be used, non-limiting mention may be made of silicone polymers grafted with a hydrocarbon chain and hydrocarbon polymers grafted with a silicone chain.

Thus, block or graft block copolymers comprising at least one block of polyorganosiloxane type and at least one block of a radical polymer can be used, such as graft copolymers of acrylic/silicone type, which can be used for example, when the non-aqueous medium is silicone-based.

It is also possible to use block or graft block copolymers comprising at least one block of polyorganosiloxane type and at least one polyether. The polyorganopolysiloxane block can be, for example, a polydimethylsiloxane or alternatively a poly(C₂-C₁₈)-alkylmethylsiloxane; the polyether block can be a poly(C₂-C₁₈)alkylene, such as polyoxyethylene and/or polyoxypropylene. For instance, it is possible to use dimethicone copolyols or (C₂-C₁₈)alkyldimethicone copolyols such as those sold under the name Dow Corning 3225C by the company Dow Corning, and lauryl methicones such as those sold under the name Dow Corning Q2-5200 by the company Dow Corning.

Block or graft block copolymers which may also be mentioned are those comprising at least one block resulting from the polymerization of at least one ethylenic monomer comprising at least one optionally conjugated ethylenic bond, such as ethylene, or dienes, such as butadiene and isoprene, and at least one block of a vinyl polymer, for example, a styrenic polymer. When the ethylenic monomer contains at least one optionally conjugated ethylenic bond, the residual ethylenic unsaturations after the polymerization are generally hydrogenated. Thus, in a known manner, the polymerization of isoprene leads, after hydrogenation, to the formation of ethylene-propylene block, and the polymerization of butadiene leads, after hydrogenation, to the formation of ethylene-butylene block. Among these polymers, non-limiting mention may be made of block copolymers, for instance of diblock or triblock type such as polystyrene/polyisoprene (SI), polystyrene/polybutadiene (SB), such as those sold under the name Luvitol HSB by BASF, polystyrene/copoly-(ethylene-propylene) (SEP), such as those sold under the name Kraton by Shell Chemical Co., or alternatively polystyrene/copoly(ethylene-butylene) (SEB). For example, Kraton G1650 (SEBS), Kraton G1651 (SEBS), Kraton G1652 (SEBS), Kraton G1657X (SEBS), Kraton G1701 X (SEP), Kraton G11702X (SEP), Kraton G1726X (SEB), Kraton D-1101 (SBS), Kraton D-1102 (SBS) or Kraton D-1107 (SIS) may be used. The polymers are generally referred to as copolymers of hydrogenated or non-hydrogenated dienes. Gelled Permethyl 99A-750, 99A-753-59 and 99A-753-58 (mixture of triblock polymer and star polymer), Versagel 5960 from Penreco (triblock polymer+star polymer), and OS129880, OS129881 and OS84383 from Lubrizol (styrene/methacrylate copolymer) can also be used.

As block or graft block copolymers comprising at least one block resulting from the polymerization of at least one ethylenic monomer containing at least one ethylenic bond and at least one block of an acrylic polymer, non-limiting mention may be made of poly(methyl methacrylate)/polyisobutylene diblock or triblock copolymers, or graft copolymers comprising a poly(methyl methacrylate) backbone and polyisobutylene grafts.

As block or graft block copolymers comprising at least one block resulting from the polymerization of at least one ethylenic monomer containing at least one ethylenic bond and at least one block of a polyether such as a C₂-C₁₈ polyalkylene, for instance, polyethylenated and/or polyoxypropylenated, non-limiting mention may be made of polyoxyethylene/polybutadiene or polyoxyethylene/polyisobutylene diblock or triblock copolymers.

When a random polymer is used as stabilizer, it is selected such that it has a sufficient amount of groups making it soluble in the intended synthesis solvent.

It is thus possible to use copolymers based on alkyl acrylates or methacrylates derived from C₁-C₄ alcohols and alkyl acrylates or methacrylates derived from C₈-C₃₀ alcohols. Non-limiting mention may be made, for example, of stearyl methacrylate/methyl methacrylate copolymer.

When the polymer synthesis solvent is apolar, for example, it is possible to select as stabilizer a polymer which gives the most complete coverage possible of the particles, a plurality of stabilizing polymer chains then being adsorbed onto a polymer particle obtained by polymerization. In this case, for instance, it is possible to use as stabilizer either a graft polymer or a block polymer, so as to have better interfacial activity. Without being bound by theory, it is believed that blocks or grafts which are insoluble in the synthesis solvent give a more voluminous coverage at the surface of the particles.

When the liquid synthesis solvent comprises at least one silicone oil, the stabilizer may be chosen for example, from block and graft block copolymers comprising at least one block of polyorganosiloxane type and at least one block of a radical polymer or of a polyether or a polyester such as polyoxypropylenated and/or oxyethylenated blocks.

When the liquid fatty phase comprises no silicone oil, the stabilizer may be chosen from, for example:

(a) block or graft block copolymers comprising at least one block of polyorganosiloxane type and at least one block chosen from radical polymers, polyethers, and polyesters,

(b) copolymers of alkyl acrylates and/or methacrylates derived from C₁-C₄ alcohols, and of alkyl acrylates and/or methacrylates derived from C₈-C₃₀ alcohols,

(c) block or graft block copolymers comprising at least one block resulting from the polymerization of at least one ethylenic monomer containing conjugated ethylenic bonds, and at least one block of a vinyl or acrylic polymer or of a polyether or a polyester, or mixtures thereof.

For example, diblock polymers may be used as stabilizers.

Plasticizer in the First Composition

The polymers in dispersion that can be used in the first composition of the present disclosure can have, for example, a glass transition temperature (T_{g}) ranging from −100° C. to 300° C., such as from −50° C. to 100° C., for instance, from −10° C. to 50° C.

In the context of the present disclosure, a plasticizer for the particular polymer in the first composition is added to the polymer particles in dispersion so as to lower the T_{g} of the polymer film and to increase the adhesion of the polymer film to its substrate, such as the keratin materials. The plasticizer lowers, for example, the glass transition temperature of the polymer by at least 1, 2, 3 or 4° C., and for instance, lowers the temperature by an amount ranging from 5° C. to 20° C. In one aspect of the present disclosure, the plasticizer lowers, for instance, the glass transition temperature of the polymer by at least 1, 2, 3 or 4° C., for example lowering the glass transition temperature in an amount ranging from 5° C. to 20° C., when the plasticizer is not present in an amount of more than 10% by weight of the polymer.

In one aspect of the present disclosure, the polymer plasticizer is selected from plasticizers having a solubility parameter δₕ ranging from 5.5 to 11, such as, from 5.9 to 11, for instance, from 7 to 10.5, such as from 8 to 10, and for example, from 9 to 10 (J/cm³)^{1/2}.

The solubility parameter δₚ of the plasticizer for example, ranges from 1.5 to 4.5, such as from 1.5 to 4, for instance from 1.5 to 3.5, and for further example ranging from 2 to 3 (J/cm³)^{1/2}.

The plasticizer can be, for example, liquid at ambient temperature and atmospheric pressure. It can be, for example tributoxyethyl phosphate.

The definition of the solubility parameters according to Hansen is well known to the person skilled in the art and is described for example, in the article by C. M. Hansen: “The three dimensional solubility parameters” J. Paint Technol. 39, 105 (1967). These parameters are also described in JP-A-08-109121 from Kao, and in the document by D. W. Van Krevelen “Properties of polymers” (1990), p. 190.

According to Hansen:

δₚ characterizes the Debye interaction forces between permanent dipoles; and

δₕ characterizes the specific interaction forces, e.g., of hydrogen bonds, acid/base, donor/receptor type, etc.

The parameters δₚ and δₕ are generally expressed in (J/cm³)^{1/2}. They are determined at ambient temperature (25° C.) and for example, according to the calculation method indicated in the above-cited Kao patent.

In the composition of the present disclosure, it is possible to use a mixture of plasticizers satisfying the above conditions. In this case the solubility parameters of the mixture are determined from those of the plasticizers taken separately, according to the following relationships:

δₚₘᵢₓ=Σxi δₚᵢ and δₕₘᵢₓ=Σxi δₕᵢ

wherein xi is the volume fraction of the plasticizer (i) in the mixture.

It is within the scope of the person skilled in the art to determine the amounts of each plasticizer so as to obtain a mixture of plasticizers meeting the above conditions.

In one aspect of the present disclosure, the plasticizer may be selected from esters of at least one carboxylic acid containing 1 to 7 carbon atoms and a polyol containing at least 4 hydroxyl groups.

The polyol according to the present disclosure may be a monosaccharide—a polyhydroxyaldehyde (aldose) or polyhydroxyketone (ketose)—which may be cyclized or not. The polyol may be, for example, a monosaccharide cyclized in hemiacetal form. The polyol may also be a polyol derived from a monosaccharide, such as erythritol, xylitol or sorbitol.

Among aldoses, non-limiting mention may be made of D-ribose, D-xylose, L-arabinose, D-glucose (or alpha-D-glucopyranose when in cyclic hemiacetal form), D-mannose and D-galactose.

Among ketoses, non-limiting mention may be made of D-xylulose and D-fructose (or beta-D-fructofuranose when in cyclic hemiacetal form).

The polyol may be chosen from monosaccharides and polysaccharides containing from 1 to 10 monosaccharide units, such as from 1 to 4, for instance, 1 to 2 monosaccharide units. For example, the polyol may be chosen from erythritol, xylitol, sorbitol, glucose and sucrose.

The polyol according to the present disclosure can be, for example, a disaccharide. Among disaccharid es, non-limiting mention may be made of sucrose, such as alpha-D-glucopyranosyl-(1-2)-beta-D-fructofuranose; lactose, such as beta-D-galactopyranosyl-(1-4)-beta-D-glucopyranose; and maltose, such as alpha-D-glucopyranosyl-(1-4)-beta-D-glucopyranose.

The polyol may be a polysaccharide composed of two or more identical monosaccharide units or at least two different monosaccharide units. The ester according to the present disclosure may be composed of a polyol substituted by at least two different monocarboxylic acids, or by at least three different monocarboxylic acids.

The ester may be obtained by copolymerizing two esters according to the present disclosure, for example by copolymerizing i) a sucrose substituted by benzoyl groups and ii) a sucrose substituted by acetyl and/or isobutyryl groups.

The ester may be obtained from at least two different monocarboxylic acids. The acid may be, for example, a monocarboxylic acid chosen from, for instance, acids comprising 1 to 7 carbon atoms, such as 1 to 5 carbon atoms, for example acetic, n-propanoic, isopropanoic, n-butanoic, isobutanoic, tert-butanoic, n-pentanoic and benzoic acids.

In one aspect of the present disclosure, the acid is an unsubstituted linear or branched acid. The acid can be, for example, chosen from acetic acid, isobutyric acid and benzoic acid.

In one aspect of the present disclosure, the ester is sucrose diacetate hexa(2-methylpropanoate).

In another aspect of the present disclosure, the plasticizer as disclosed herein is chosen from esters of aliphatic and aromatic polycarboxylic acid,s and aliphatic or aromatic alcohols comprising from 1 to 10 carbon atoms.

The aliphatic and aromatic alcohols can comprise from 1 to 10 carbon atoms, such as from 1 to 8, for instance, 1 to 6. They may be chosen from alcohols R1OH such that R1 is chosen from methyl, ethyl, propyl, isopropyl, butyl, hexyl, ethylhexyl, decyl, isodecyl, and benzyl substituted by an alkyl comprising 1 to 6 carbon atoms.

The aliphatic and aromatic polycarboxylic acids may comprise for example, from 3 to 12 carbon atoms, for instance, from 3 to 10 carbon atoms, such as from 3 to 8 carbon atoms, for further example from 6 to 8 carbon atoms. The aliphatic and aromatic polycarboxylic acids may be, for example, dicarboxylic acids or tricarboxylic acids.

Among dicarboxylic acids, non-limiting mention may be made of those corresponding to the formula HOOC—(CH₂)ₙ—COOH wherein n ranges from 1 to 10, such as from 2 to 8, for example 2, 4, 6 or 8. For further example, the dicarboxylic acids may be chosen from succinic, adipic and sebacic acids. The dicarboxylic acids may also be chosen from phthalic acid and its derivatives, for instance butyl benzyl phthalate, dibutyl phthalate, diethylhexyl phthalate, diethyl phthalate and dimethyl phthalate.

Among tricarboxylic acids, non-limiting mention may be made of the triacids corresponding to the formula

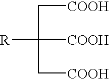

wherein R is a hydrocarbon radical, such as H, —OH and —OCOR′, wherein R′ is an alkyl comprising, for example, from 1 to 6 carbon atoms. The tricarboxylic acids may be selected for instance, from acetylcitric acid and its derivatives.

Among the citrates, non-limiting mention may be made of tributyl acetylcitrate, triethyl acetylcitrate, triethylhexyl acetylcitrate, trihexyl acetylcitrate, trihexyl butyroylcitrate, isodecyl citrate, isopropyl citrate, tributyl citrate and triethylhexyl citrate. In one aspect of the present disclosure, the carboxylic acid is not tributyl acetylcitrate.

Among adipates, non-limiting mention may be made of dibutyl adipate and di-2-ethylhexyl adipate. In one aspect of the present disclosure, the plasticizer as disclosed herein, is not diisopropyl adipate.

Among sebacates, non-limiting mention may be made of dibutyl sebacate, diethylhexyl sebacate, diethyl sebacate and diisopropyl sebacate.

Among succinates, non-limiting mention may be made of diethylhexyl succinate and diethyl succinate.

The plasticizer may be, for example, chosen from compounds having a molecular mass of less than 5,000 g/mol, such as less than 2,000, for instance, less than 1,000, for further instance, less than 900 g/mol. For example, the molecular mass of the plasticizer can be greater than 100 g/mol.

For example, the plasticizer does not comprises a polar group, such as a hydroxyl group. “Polar groups” are, for example, ionic or non-ionic polar groups chosen from —COOH; —OH; ethylene oxide; propylene oxide; —PO₄; —NHR; —NR₁R₂, with R₁ and R₂ optionally forming a ring and each being chosen from linear and branched C₁ to C₂₀ alkyl and alkoxy radicals.

The plasticizer can be present in the first composition, for example, in an amount ranging from 0.1 to 25% by weight, for instance from 0.5 to 15% by weight, such as from 3 to 15% by weight, relative to the total weight of the first composition.

The mass ratio between the polymer particles and the plasticizer for example, can range from 0.5 to 100, such as from 1 to 50, for instance, from 1 to 10, for further example, from 1 to 5.

Fatty Phase of the First Composition

The liquid fatty phase of the first composition may be composed of any cosmetically or dermatologically acceptable, and more generally physiologically acceptable, oil, chosen from, for example, oils of mineral, vegetable, and synthetic origin, including carbon-based oils, hydrocarbon oils, fluoro oils and/or silicone oils, alone or in a mixture, insofar as they form a homogeneous and macroscopically stable mixture and they are compatible with the intended use.

The total liquid fatty phase of the first composition may represent from 5 to 90% of the total weight of the composition, and for example, from 20 to 85%. For instance, the total liquid fatty phase may be present in an amount of at least 30% of the total weight of the composition. For further instance, the fatty phase may include at least one volatile oil.

Volatile Oils of the Fatty Phase

In accordance with the present disclosure, at least one volatile oil may be used These volatile oils may be hydrocarbon oils or silicone oils optionally comprising alkyl or alkoxy groups pendantly or at the end of the silicone chain.

As volatile silicone oils which can be used according to the present disclosure, non-limiting mention may be made of linear or cyclic silicones having a viscosity at ambient temperature of less than 8 cSt, and having for example, from 2 to 7 silicon atoms, these silicones optionally comprising alkyl or alkoxy groups having from 1 to 10 carbon atoms. As volatile silicone oils that can be used as disclosed herein, non-limiting mention may be made of, for example: octamethylcyclotetrasiloxane, decamethylcyclopentasiloxane, dodecamethylcyclohexasiloxane, heptamethyl-hexyltrisiloxane, heptamethyloctyltrisiloxane, octamethyltrisiloxane, decamethyltetrasiloxane and mixtures thereof.

As other volatile oils that can be used as disclosed herein, non-limiting mention may be made, for instance of C₈-C₁₆ isoalkane oils, also called isoparaffins, such as isododecane, isodecane, isohexadecane and, for example, the oils sold under the trade names Isopar and Permethyl, and for instance, isododecane (Permethyl 99A).

The volatile oil may be present in the first composition in an amount ranging from 20 to 90% by weight, relative to the total weight of the first composition, such as from 30 to 80% and for instance, from 40 to 70%.

Synthesis Solvent of the Polymer Particles

The polymer dispersion may be prepared as described in EP-A-749 747.

A mixture is prepared comprising the initial monomers and a free-radical initiator. This mixture is dissolved in a solvent, which, for the remainder of the present description, is referred to as the “synthesis solvent.” When the fatty phase is a non-volatile oil, the polymerization may be carried out in an apolar organic solvent (synthesis solvent) and then the non-volatile oil, which must be miscible with the synthesis solvent, is added and the synthesis solvent is distilled off selectively.

A synthesis solvent is selected such that the initial monomers and the free-radical initiator are soluble therein, and the polymer particles obtained are insoluble therein, so that they precipitate from the solvent as they are formed. The synthesis solvent may be, for example, chosen from alkanes such as heptane, isododecane and cyclohexane.

When the fatty phase chosen is a volatile oil, the polymerization may be carried out directly in the oil, which hence also acts as the synthesis solvent. The monomers must also be soluble therein, and the free-radical initiator too, and the polymer obtained must be insoluble therein.

The monomers can be, for example, present in the synthesis solvent, prior to polymerization, in an amount ranging from 5-20% by weight of the reaction mixture. The total amount of the monomers may be present in the solvent before the beginning of the reaction, or a fraction of the monomers may be added at the rate at which the polymerization reaction proceeds.

The free-radical initiator may be, for example, chosen from azobisisobutyronitrile and tert-butyl peroxy-2-ethylhexanoate.

The volatile phase of the composition may be composed of the synthesis solvent of the dispersed polymer particles.

Non-Volatile Oil of the Fatty Phase

The fatty phase may comprise, for example, at least one apolar or relatively non-polar non-volatile oil.

As relatively non-polar non-volatile oils which can be used according to the present disclosure, non-limiting mention may be made of apolar oils or, for example, oils comprising an alkyl chain, such as a C₃-C₄₀ alkyl chain. Non-limiting examples of apolar or relatively non-polar oils include the following:

linear or branched hydrocarbons such as liquid paraffin, vaseline oil and light naphthalene oil and hydrogenated polyisobutene;

hydrocarbon oils of animal origin such as squalene;

vegetable hydrocarbon oils such as the liquid triglycerides of fatty acids of at least 10 carbon atoms;

synthetic esters and ethers, for example, of fatty acids, such as the oils of formula R₁CO(O)ₓR₂ wherein R₁ is chosen from the residue of an acid comprising from 2 to 29 carbon atoms, with x being equal to 0 or 1, and R₂ is chosen from hydrocarbon chains comprising from 3 to 30 carbon atoms, such as, for example, tributyl acetylcitrate, oleyl erucate, 2-octyldodecyl behenate, triisoarachidyl citrate, isocetyl stearoylstearate, octyldodecanyl stearoylstearate, n-propyl acetate, tridecyl trimellitate, dodecane dioleate, diisocetyl stearate, arachidyl propionate, dibutyl phthalate, propylene carbonate, octyldodecyl pentanoate; and polyol esters such as vitamin F, sorbitan isostearate, glyceryl triisostearate and diglycerol;

silicone oils such as polydimethylsiloxanes (PDMS), optionally comprising a C₃-C₄₀ alkyl or alkoxy chain or a phenyl chain, such as phenyltrimethicones, polyalkylmethylsiloxanes, optionally fluorinated such as the polymethyltrifluoropropyldimethylsiloxanes, or with functional groups such as hydroxyl, thiol and/or amine groups; polysiloxanes modified with fatty acids, fatty alcohols or polyoxyalkylenes;

fluoro oils;

mixtures thereof.

These non-volatile apolar or relatively non-polar oils may be present in the first composition in an amount ranging from 0.1 to 20% by weight, relative to the total weight of the first composition, such as from 0.5 to 10% for instance, from 1 to 5% of the total weight of the first composition.

For example, the non-volatile oil can be apolar. For further example, it can be selected from hydrocarbons, especially alkanes, such as hydrogenated polyisobutene.

The fatty phase may also include a polar oil chosen from fatty acid esters ranging from 7 to 29 carbon atoms such as diisostearyl malate, isopropyl palmitate, diisopropyl adipate, the triglycerides of caprylic/capric acids, such as those sold by Stearineries Dubois, and those sold under the names Miglyol 810, 812 and 818 by Dynamit Nobel, the oil of shea butter, isopropyl myristate, butyl stearate, hexyl laurate, diisopropyl adipate, isononyl isononanoate, 2-hexyldecyl laurate, 2-octyldecyl palmitate, 2-octyldodecyl myristate and lactate, di-2-ethylhexyl succinate, 2-ethylhexyl palmitate, 2-octyldodecyl stearate, castor oil; esters of lanolic acid, lauric acid and stearic acid; higher fatty alcohols, i.e., those of 7 to 29 carbon atoms, such as stearyl alcohol, linoleyl alcohol and linolenyl alcohol, isostearyl alcohol, 2-octyldodecanol, decanol, dodecanol, octadecanol and oleyl alcohol; higher fatty acids, i.e., those of 7 to 29 carbon atoms, such as myristic acid, palmitic acid, stearic acid, behenic acid, oleic acid, linoleic acid, linolenic acid andisostearic acid.

These non-volatile polar oils may be present in the first composition in an amount ranging from 0.1 to 10% of the total weight of the composition, such as from 1 to 5%.

In one aspect of the present disclosure, the liquid fatty phase comprises at least one apolar volatile oil and at least one apolar non-volatile oil.

Gelling Agent

In another aspect of the present disclosure, the first composition may comprise a gelling agent for the liquid fatty phase, the agent being chosen from polymeric and mineral gelling agents.

The gelling agent may also be chosen from ethylene homopolymers and copolymers whose weight-average molecular mass ranges from 300 to 500,000, such as from 500 to 100,000 g/mol.

The gelling agent may be chosen from olefin copolymers of controlled crystallization, as described in application EP-A-1 034 776 fromL'Oréal, such as, for example, the ethylene/octene copolymer sold under the reference Engage 8400 by DuPont de Nemours.

The olefin copolymer or copolymers can be present in the first composition in an amount ranging from 0.5 to 20% by weight, such as, from 1 to 10% by weight, relative to the total weight of the first composition.

In another aspect of the present disclosure, a polycaprolactone is used as gelling agent. The polycaprolactone for example, can be chosen from E-caprolactone homopolymers. Homopolymerization may be initiated with a diol, for instance, a diol having from 2 to 10 atoms, such as diethylene glycol, 1,4-butanediol and neopentyl glycol.

It is possible, for example, to use polycaprolactones with a molecular weight ranging from 300 to 2,000 g/mol, such as those sold under the name Capa® 2125 by Solvay. The polycaprolactone may be present in the first composition in an amount ranging from 0.1% to 30% by weight, relative to the total weight of the first composition, for instance, from 0.5% to 25% by weight, such as from 1% to 20% by weight, and for further example, from 3% to 15% by weight.

In another aspect of the present disclosure, the gelling agent present in the composition according to the present disclosure, is an amorphous polymer formed by polymerizing an olefin as described in application EP 1 002 528, the content of which is incorporated into the present application by reference. The olefin may be, for example, an elastomeric ethylenically unsaturated monomer.

As examples of olefins, non-limiting mention may be made of ethylenic hydrocarbon monomers, having for example, one or two ethylenic unsaturations and having from 2 to 5 carbon atoms, such as ethylene, propylene, butadiene and isoprene.

An amorphous polymer is a polymer which does not have a crystalline form. The amorphous polymeric gelling agent may be for example, chosen from diblock, triblock, multiblock, radial and star copolymers, such as triblock, multiblock, radial and star copolymers. Polymeric gelling agents of this kind are described in application U.S. Application A2002/005562 and in U.S. Pat. No. 5,221,534.

The polymeric gelling agent may be for example, an amorphous block copolymer of styrene and an olefin. The polymeric gelling agent may also be, for example, hydrogenated in order to reduce the ethylenic unsaturations that remain after the polymerization of the monomers.

For instance, the polymeric gelling agent may be an optionally hydrogenated copolymer comprising styrene blocks and ethylene/C₃-C₄ alkylene blocks. The polymeric gelling agent may be, for example a triblock copolymer, for further example, hydrogenated, chosen from styrene-ethylene/propylene-styrene copolymers, styrene-ethylene/butadiene-styrene copolymers, styrene-isoprene-styrene copolymers and styrene-butadiene-styrene copolymers. Triblock polymers are sold for instance, under the names Kraton® G1650E, Kraton® G1652, Kraton® D1101, Kraton® Dl 102 and Kraton® D1160 by Kraton.

The amorphous polymeric gelling agent may be present in the first composition in an amount ranging from 0.05% to 5% by weight, relative to the total weight of the first composition, such as ranging from 0.1% to 3% by weight, and for instance ranging from 0.2% to 2% by weight.

The polymer particles dispersed in the organic phase and the polymeric gelling agent can be, for example, present in the first composition in an amount such that the weight ratio of dispersed and surface-stabilized polymer particles to polymeric gelling agent ranges from 10 to 30, such as from 15 to 25, for instance, from 18 to 22. In anotheraspect of the present disclosure, the gelling agent may be chosen from, for example:

pyrogenic silica, optionally hydrophobically treated on its surface, with a particle size of less than 10 microns; and

optionally modified clays, such as hectorites modified with a C₁₀ to C₂₂ fatty acid ammonium chloride, such as hectorite modified with distearyldimethylammonium chloride.

As organic-phase gelling agents, non-limiting mention may also be made of the following:

partly or totally crosslinked elastomeric organopolysiloxanes of three-dimensional structure, such as those sold under the name KSG6, KSG16 and KSG18 by Shin-Etsu, Trefil E-505C and Trefil E-506C by Dow Corning, Gransil SR-CYC, SR DMF10, SR-DC556, SR 5CYC gel, SR DMF 10 gel and SR DC 556 gel by Grant Industries, and SF 1204 and JK 113 by General Electric;

galactomannans comprising from 1 to 6, for example, 2 to 4 hydroxyl groups per monosaccharide unit, which are substituted by a saturated or unsaturated alkyl chain, such as guar gum alkylated with C₁ to C₆ for instance C₁ to C₃ alkyl chains, and for further example, the ethylated guar having a degree of substitution ranging from 2 to 3, such as that sold by Aqualon under the name N-Hance-AG;

gums, for instance, silicone gums, such as PDMS having a viscosity greater than 100,000 cSt, and mixtures thereof.

Colloidal Dispersion of the First Composition

The first composition according to the present disclosure may, for example, comprise particles that are solid at ambient temperature and are dispersed in the physiologically acceptable medium, and which are introduced into the composition in the form of a colloidal dispersion, also referred to as a “particle paste,” as described in application WO 02/39961, the content of which is incorporated by reference into the present application.

A colloidal dispersion, or “particle paste,” for the purposes of the present disclosure, is a concentrated colloidal dispersion of coated or uncoated particles in a continuous medium, this dispersion being stabilized using a dispersant or, where appropriate, without a dispersant. These particles may be chosen from pigments, nacres, and solid fillers. These particles may be of any form, for example, spherical or elongated such as fibers. They are insoluble in the medium.

The purpose of the dispersant is to protect the dispersed particles against agglomeration or flocculation. The concentration of dispersant used to stabilize a colloidal dispersion may range, for example, from 0.3 to 5 mg/m², such as, from 0.5 to 4 mg/m² of particle surface area. The dispersant may be chosen from at least one of surfactants, oligomers, and polymers, and carries at least one functionality that has a high affinity for the surface of the particles to be dispersed. For example, the dispersants are able to attach physically or chemically to the surface of the particles. The dispersants additionally exhibit at least one functional group that is compatible with or soluble in the continuous medium. Use can be made, for example, of esters of 12-hydroxystearic acid, sfor instance of C₈ to C₂₀ fatty acids and polyols, such as glycerol and diglycerol, non-limiting examples include poly(12-hydroxystearic) stearate with a molecular weight of approximately 750 g/mol, such as that sold under the name Solsperse 21 000 by Avecia; polyglyceryl-2 dipolyhydroxystearate (CTFA name), sold under the reference Dehymuls PGPH by Henkel; and also polyhydroxystearic acid, such as that sold under the reference Arlacel P100 by Uniqema, and mixtures thereof.

Among other dispersants that can be used in the first composition as disclosed herein, non-limiting mention may be made of quaternary ammonium derivatives of polycondensed fatty acids, such as Solsperse 17 000 sold by Avecia, and the polydimethylsiloxane/oxypropylene mixtures such as those sold by Dow Corning under the references DC2-5185 and DC2-5225 C.

Polydihydroxystearic acid and the esters of 12-hydroxystearic acid for example, may be used for a hydrocarbon-based or fluorine-based medium, while the dimethylsiloxane oxyethylene/oxypropylene mixtures may be, for instance, used for a silicone-based medium.

The colloidal dispersion is a suspension of particles generally of micron size, e.g., less than 10 μm, in a continuous medium. The volume fraction of particles in a concentrated dispersion can range from 20 to 40%, for example, greater than 30%, corresponding to a weight fraction which can range up to 70%, depending on the density of the particles.

The particles dispersed in the medium may be chosen from mineral particles and organic particles, such as those described below.

The continuous medium of the paste may be any medium and may comprise any solvent or liquid fatty substance and mixtures thereof. For example, the liquid medium of the particle paste may be one of the liquid fatty substances or oils that is also desired to be used in the first composition, hence forming part of the liquid fatty phase.

The “particle paste” or colloidal dispersion may also be, for example, a “pigment paste” comprising a colloidal dispersion of colored, coated or uncoated, particles. These colored particles may be chosen from at least one of pigments and nacres.

The colloidal dispersion may be present in the first composition for example, in an amount ranging from 0.5 to 60% by weight, relative to the total weight of the first composition, such as from 2 to 40%, and for instance, preferably from 2 to 30%.

The pigments may be chosen from white, colored, mineral, organic, interference, and non-interference pigments. Among mineral pigments, non-limiting mention may be made of titanium dioxide, optionally with surface treatment, zirconium oxide, cerium oxide, zinc oxide, iron oxide (black, yellow or red) chromium oxide, manganese violet, ultramarine blue, chromium hydrate and ferric blue. Among organic pigments, non-limiting mention may be made of carbon black, organic lake pigments of barium, strontium, calcium and aluminium, including those certified by the U.S. Food and Drug Administration (FDA) for example, D&C and FD&C, and those exempt from FDA certification, such as lakes based on cochineal carmine. The pigments may be present in the first composition in an amount ranging from 0.1 to 50% by weight of active material, such as from 0.5 to 35%, for instance, from 2 to 25%, relative to the total weight of the first composition.

The nacreous pigments may be chosen from white nacreous pigments such as titanium-covered mica or bismuth oxychloride; colored nacreous pigments such as titanium mica with iron oxides, titanium mica with, for example, ferric blue and chromium oxide; titanium mica with an organic pigment of the aforementioned typ, and also nacreous pigments based on bismuth oxychloride. The nacreous pigments, may be present in the first composition in an amount ranging from 0 to 25% by weight of active material, relative to the total weight of the composition, such as from 0.1 to 15%. Use may therefore be made of pigments having goniochromatic properties and of metallic effect pigments, such as those described in the application filed under number FR 0 209 246, the content of which is hereby incorporated into the present application.

The fillers may be chosen from mineral, organic, lamellar, and spherical fillers. Non-limiting mention may be made of talc, mica, silica, kaolin, Nylon® powders, such as Orgasol® from Atochem, poly-β-alanine powders, polyethylene powders, powders of polymers of tetrafluoroethylene, such as Teflon®, lauroyllysine, starch, boron nitride, hollow microspheres such as Expancel® sold by Nobel Industries, Polytrap® sold by Dow Corning, and silicone resin microbeads, such as Tospearls® from Toshiba, for example, precipitated calcium carbonate, magnesium carbonate and bicarbonate, hydroxyapatite, hollow silica microspheres, such as Silica Beads® from Maprecos, glass and ceramic microcapsules, and metal soaps derived from organic carboxylic acids having from 8 to 22 carbon atoms, for instance from 12 to 18 carbon atoms, for example zinc stearate, magnesium stearate and lithium stearate, zinc laurate and magnesium myristate.

Wax of the First Composition

Depending on the type of application envisaged, the first composition according to the present disclosure may further comprise at least one wax.

A wax for the purposes of the present disclosure is a lipophilic fatty compound that is solid at ambient temperature (25° C.) and exhibits a reversible solid/liquid state change, the compound having a melting temperature of greater than 40° C. and possibly up to 200° C., and having an anisotropic crystalline organization in the solid state. The size of the crystals is such that they diffract and/or scatter light, thereby endowing the composition with a turbid, more or less opaque appearance. By bringing the wax to its melting temperature it can be made miscible with the oils and made to form a microscopically homogeneous mixture; however, by bringing the temperature of the mixture back to ambient temperature, the wax is recrystallized in the oils of the mixture. Without being bound by theory, it is believed that this recrystallization in the mixture is responsible for the decrease in the gloss of the mixture.

In one aspect of the present disclosure, for example, linear hydrocarbon waxes may be used. Their melting point can be for instance, greater than 35° C., for example greater than 55° C., such as greater than 80° C.

The linear hydrocarbon waxes may be chosen from, for example, substituted linear alkanes, unsubstituted linear alkanes, unsubstituted linear alkenes, and substituted linear alkenes, an unsubstituted compound being composed solely of carbon and hydrogen, and wherein the substituents mentioned above do not contain any carbon atoms.

Linear hydrocarbon waxes include polymers and copolymers of ethylene with a molecular weight ranging from 400 to 800, for example Polywax 500 and Polywax 400 sold by New Phase Technologies. Linear hydrocarbon waxes also include linear paraffin waxes, such as the paraffins S&P 206, S&P 173 and S&P 434 from Strahl & Pitsch. Linear hydrocarbon waxes further include long-chain linear alcohols, such as the products comprising a mixture of polyethylene and alcohols comprising 20 to 50 carbon atoms, for instance, Performacol 425 and Performacol 550, i.e., a mixture in 20/80 proportions, sold by New Phase Technologies.

The waxes may be present in the first composition in an amount ranging from 2 to 30% by weight, relative to the total weight of the first compostion, such as from 5 to 20%, such as from 5 to 15%, so as not excessively to diminish the gloss of the composition and of the film deposited on the lips and/or skin.

Second Composition

According to one aspect of the present disclosure, the second composition as disclosed herein, may comprise at least one apolar or relatively non-polar compound, which may be chosen from oils, gums and/or waxes. The second composition may comprise, for example, more than 70%, such as more than 80% by weight, for instance, 100% by weight of apolar or relatively non-polar compounds. These apolar or relatively non-polar compounds may be chosen from, for example, silicone compounds, colorants and gelling agents, and necessarily comprise the high molecular weight polymer.

The second composition may be, for example, one of the compositions described in U.S. Application No. 60/375,814, the content of which is hereby incorporated into the present application.

In one aspect of the present disclosure, the second composition comprises a physiologically acceptable medium which comprises a liquid phase which is not volatile at ambient temperature and atmospheric pressure.

The second composition may comprise, for example, 15 to 90% by weight of at least one non-volatile liquid phase, relative to the total weight of the second composition.

A “non-volatile liquid phase” is any medium apt to remain on the skin or the lips for several hours. A non-volatile liquid phase has for example, a non-zero vapor pressure at ambient temperature and atmospheric pressure, of less than 0.02 mm Hg

Pa), such as less than 10⁻³ mm Hg (0.13 Pa).

The non-volatile liquid phase of the second composition may be, for example, apolar or relatively non-polar and for instance, can comprise at least one oil chosen from hydrocarbon oil, one silicone oil and/or one liquid fluoro oil.

The non-volatile oils may be chosen from polydimethylsiloxanes; alkyldimethicones; polyphenylmethylsiloxanes, such as phenyldimethicones and phenyl-trimethicones; and silicones modified with aliphatic and/or aromatic groups, optionally fluorinated, or with functional groups such as hydroxyl, thiol and/or amine groups.

Among non-volatile oils, non-limiting mention may be made, for example, of an oil chosen from the silicones of formula (I):

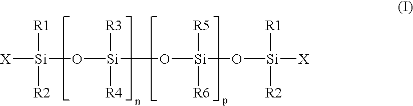

wherein:

R₁, R₂, R₅ and R₆, which may be identical or different, may be chosen from alkyl radicals having 1 to 6 carbon atoms,

R₃ and R₄, which may be identical or different, are chosen from alkyl radicals having 1 to 6 carbon atoms and aryl radicals,

X can be chosen from alkyl radicals having 1 to 6 carbon atoms, a hydroxyl radical and vinyl radicals,

n and p are chosen so as to give the oil a weight-average molecular mass of less than 200,000 g/mol, such as less than 150,000 g/mol, for instance, less than 100 000 g/mol.

The non-volatile silicone of formula (I) maybe, for example, a polydimethylsiloxane with a viscosity ranging from 0.5 to 10,000 cSt, for instance, ranging from 0.5 to 1,000 cSt, for further example, DC 200 with a viscosity of 350 cSt, sold by Dow Corning.

The non-volatile liquid phase of the second composition may comprise, for example, at least one fluoro oil of formula (II):

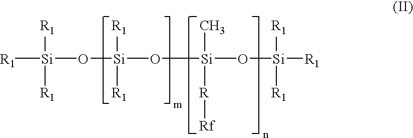

wherein:

R is chosen from linear and branched alkylenyl groups having 1 to 6 carbon atoms, such as a divalent methylenyl, ethylenyl, propylenyl and butylenyl group,

Rf is chosen from fluoroalkyl radicals, such as a perfluoroalkyl radical, having 1 to 9 carbon atoms, for instance, 1 to 4 carbon atoms,

R₁, which may be identical or different at each occurrence, is chosen from C₁-C₂₀, such as C₁-C₄, alkyl radicals, a hydroxyl radical and a phenyl radical,

m is an integer ranging from 0 to 150, such as from 20 to 100, and

n is an integer ranging from 1 to 300, such as from 1 to 100.

In one aspect of the present disclosure, the non-volatile liquid phase of the second composition comprises oils of formula (II) wherein R₁ is a methyl, R is an ethyl and Rf is CF₃. Among tluorosilicone compounds of formula (II), non-limiting mention may be made, for example, of those sold by Shin Etsu under the names X22-819, X22-820, X22-821 and X22-822 or else FL-100.

Among fluoro oils, non-limiting mention may also be made of fluorinated polyethers selected from the compounds of formula (III) as follows:

R₆—(CF₂—CFR₃—CF₂O)ₚ—(CFR₄—CF₂—O)_{q}—(CFR₅—O)ᵣR₇ (III)

wherein:

R₃ to R₆, which may be identical or different, are monovalent radicals chosen from —F, —(CF₂)ₙ—CF₃ and —O—(CF₂)ₙ—CF₃,

R₇ is a monovalent radical chosen from —F and —(CF₂)ₙ—CF₃,

n is an integer ranging from 0 to 4,

p is an integer ranging from 0 to 600, q is an integer ranging from 0 to 860 and r is an integer ranging from 0 to 1 500, wherein p, q and r are selected such that the weight-average molecular mass of the compound ranges from 500 to 100 000, such as from 500 to 10 000.

The fluoro oils may also be chosen from fluorinated alkanes selected in turn from C₂-C₅₀, such as C₅-C₃₀, fluoroalkanes and perfluoroalkanes, such as perfluorodecalin, perfluoroadamantane and bromoperfluorooctyl and mixtures thereof.

High Molecular Weight Polymer of the Second Composition

When the second composition according to the present disclosure is liquid, the high molecular weight polymer is present in an amount ranging, for example, from 20 to 50% by weight relative to the total weight of the second composition.

When the second composition according to the present disclosure is solid, the high molecular weight polymer is present in an amount ranging from, for example, 2 to 40% by weight, relative to the total weight of the composition.

The high molecular weight polymer may be, for example, a silicone polymer.

The high molecular weight polymer may be liquid or solid at ambient temperature and its weight-average molecular mass is greater than or equal to 200,000 g/mol, for example, ranging from 200,000 to 4,000,000, such as from 200,000 to 2,500,000, such as from 200,000 to 2,000,000 g/mol.

The viscosity of this polymer may range from 1,000 to 10,000,000 cSt, such as from 100,000 to 1,000,000 cSt, for instance, from 300,000 to 700,000 cSt, measured in accordance with standard ASTM D-445.

The high molecular weight polymer may be, for example, an ungrafted polymer, i.e. a polymer obtained by polymerizing at least one monomer without subsequent reaction of the side chains with another chemical compound. The polymer may be chosen from at least one of dimethiconols, fluorosilicones, and dimethicones. The polymer may be, for instance, a homopolymer.

For example, use may be made of a high molecular weight polymer of formula (IV):

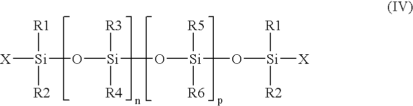

wherein:

R₁, R₂, R₅ and R₆, which may be identical or different, are chosen from alkyl radicals having 1 to 6 carbon atoms that may be optionally substituted by at least one fluorine atom,

R₃ and R₄, which may be identical or different, are chosen from alkyl radicals having 1 to 6 carbon atoms and aryl radicals,

X is chosen from alkyl radicals having 1 to 6 carbon atoms, a hydroxyl radical, vinyl and allyl radicals, and alkoxy radicasl having 1 to 6 carbon atoms, n and p being selected such that the silicone compound has a weight-average molecular mass of greater than or equal to 200,000 g/mol.

For example, p may be equal to 0.

In one aspect of the present disclosure, the second composition comprises polymers of formula (IV) wherein R₁ to R₆ are methyl groups and the substituent X is a hydroxyl group, and are thus dimethiconols. Further examples include polymers of formula (IV) wherein p is equal to 0 and n ranges from 2,000 to 40,000, such as from 3,000 to 30,000. Non-limiting mention may also be made of polymers having a molecular mass ranging from 1,500,000 to 2,000,000 g/mol.

In another aspect of the present disclosure, the high molecular weight polymer is the dimethiconol sold by Dow Corning in a polydimethylsiloxane (5 cSt) under reference D2-9085, the viscosity of the mixture being 1 550 cSt, or the dimethiconol sold by Dow Corning in a polydimethylsiloxane (5 cSt) under reference DC 1503. The dimethiconol, with a molecular weight of 1 770 000 g/mol, sold by Dow Corning under reference Q2-1403 or Q2-1401, may also be used, the viscosity of the mixture being 4 000 cSt.

Among high molecular weight polymers that can be used according to the present disclosure, non-limiting mention may be made of those wherein:

substituents R₁ to R₆ and X are methyl groups, such as that sold under the name SE30 by General Electric, and that sold under the name AK 500000 by Wacker,

substituents R₁ to R₆ and X are methyl groups, and p and n are such that the molecular weight is 250,000 g/mol, such as that sold under the name Silbione 70047 V by Rhodia,

substituents R₁ to R₆ are methyl groups and substituent X is a hydroxyl group, such as that sold under the name Q2-1401 or Q2-1403 by Dow Corning,

substituents R₁, R₂, R₅, R₆ and X are methyl groups, substituents R₃ and R₄ are aryl groups, and n and p are such that the molecular weight of the polymer is 600,000 g/mol, such as that sold under the name 761 by Rhone-Poulenc.

The high molecular weight silicone polymer may be, for example, introduced into the composition in the form of a mixture with a liquid silicone, the viscosity of the liquid silicone ranging from 0.5 to 10,000 cSt, for instance from 0.5 to 500 cSt, such as from 1 to 10 cSt.

The silicone fluid may be chosen from at least one of polyalkylsiloxanes, polyarylsiloxanes, and polyalkylarylsiloxanes. The liquid silicone may be a volatile silicone such as a cyclic polydimethylsiloxane comprising 3 to 7-(CH₃)₂SiO— units.

The liquid silicone may also be a non-volatile polydimethylsiloxane silicone, for example, with a viscosity ranging from 0.5 to 10,000 cSt, for instance, of the order of 5 cSt, for example the silicone sold under reference DC 200 by Dow Corning.

The proportion of the high molecular weight silicone polymer in the high molecular weight silicone polymer/liquid silicone mixture may, for example, range from 10/90 to 20/80. The viscosity of the high molecular weight silicone polymer/liquid silicone mixture may, for instance, range from 1,000 to 10,000 cSt.

High molecular weight dimethicones according to the present disclosure, include the dimethicones described in U.S. Pat. No. 4,152,416. They are sold for example under references SE30, SE33, SE 54 and SE 76.

Dimethicones according to the present disclosure, may be, for example, compounds of formula (III) wherein R₁ to R₆ and X are methyls and p is equal to 0. The molecular weight of these polymers may range for example, from 200,000 to 300,000, such as from 240,000 and 260,000 g/mol.

The dimethicones according to the present disclosure include polydimethylsiloxanes, (polydimethylsiloxane)(methylvinylsiloxane) copolymers, poly(dimethylsiloxane)(diphenyl)(methylvinylsiloxane) copolymers and mixtures thereof.

The high molecular weight fluorosilicones according to the present disclosure, may have, for example, a molecular weight ranging from 200,000 to 300,000, such as from 240,000 to 260,000 g/mol.

Wax in the Second Composition

The second composition may also comprise for example, at least one wax, for instance, when it is in solid form.

The waxes may be present in the second composition in an amount ranging from 2 to 30% by weight, relative to the total weight of the second composition, such as from 5 to 20%, for instance from 5 to 15%.

In one aspect of the present disclosure, the second composition may be, for example, linear hydrocarbon waxes. The melting point of the linear hydrocarbon waxes may be, for instance, greater than 35° C., for example greater than 55° C., such as greater than 80° C.

The linear hydrocarbon waxes may be chosen from, for example, substituted linear alkanes, unsubstituted linear alkanes, unsubstituted linear alkenes, substituted linear alkenes, an unsubstituted compound being composed solely of carbon and hydrogen, and the substituents mentioned above not containing any carbon atoms.

Linear hydrocarbon waxes include polymers and copolymers of ethylene with a molecular weight ranging from 400 to 800, for example Polywax 500 and Polywax 400, sold by New Phase Technologies.

Linear hydrocarbon waxes include linear paraffin waxes, such as paraffins S&P 206, S&P 173 and S&P 434 from Strahl & Pitsch.

Linear hydrocarbon waxes include long-chain linear alcohols, such as the products comprising a mixture of polyethylene and alcohols comprising from 20 to 50 carbon atoms, such as Performacol 425 and Performacol 550, i.e. a mixture in 20/80 proportions, sold by New Phase Technologies.

The second composition may comprise, for example, a silicone wax, such as a dimethicone containing alkyl groups at the chain end. These alkyl groups may have, for example, more than 18 carbon atoms, such as ranging from 20 to 50, for instance from 30 to 45 carbon atoms.

The silicone wax can correspond, for example, to the formulae (V) and (VI)

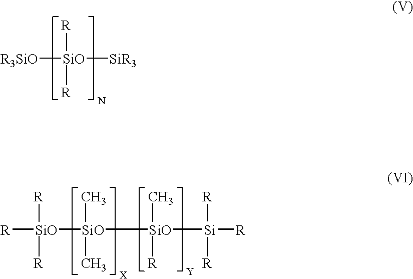

wherein R is an alkyl group, X is greater than or equal to zero, and N and Y are greater than or equal to one.

R comprises 1 to 50 carbon atoms, subject to the proviso that the compound is solid at ambient temperature.

Further, non-limiting examples of silicone waxes include:

C20-C24 alkyl methicones, C24-C28 alkyl dimethicones, C20-C24 alkyl dimethicones and C24-C28 alkyl dimethicones, sold by Archimica Fine Chemicals as SilCare 41 M40, SilCare 41 M50, SilCare 41 M70 and SilCare 41 M80,

stearyl dimethicones referenced SilCare 41 M65, sold by Archimica, or referenced DC-2503, sold by Dow Corning,

stearoxytrimethylsilanes sold as SilCare 1 M71 or DC-580,

the products Abil Wax 9810, 9800 and 2440 from Wacker-Chemie GmbH,

C30-C₄₋₅ alkyl methicones sold by Dow Corning as AMS-C30 Wax, and also C30-C45 alkyl dimethicones sold as SF1642 and SF-1632 by General Electric.

Gelling Agent

The second composition may comprise a gelling agent as described above.

Colorant

The second composition of the cosmetic product according to the present disclosure may comprise at least one colorant, which may be chosen from water-soluble dyes, fat-soluble dyes, pigments, and nacres.

Pigments may be white, colored, organic, mineral, interferential, and non-interferential particles that are insoluble in the liquid organic phase and are intended for colouring and/or opacifying the first composition.

Nacres are iridescent particles, produced for example, by certain molluscs within their shell, or else synthesized, which are insoluble in the medium of the first composition.

Dyes are compounds, such as organic compounds, which are soluble in fatty substances, such as oils, or in an aqueous-alcoholic phase.

The fat-soluble dyesthat may be used are, for example, Sudan Red, D&C Red No. 17, D&C Green No. 6, β-carotene, soya oil, Sudan Brown, D&C Yellow No. 11, D&C Violet No. 2, D&C Orange No. 5, quinoline yellow, annatto and bromo acids.

The water-soluble dyes that may be used are, for example, beet juice, methylene blue and caramel.

Among mineral pigments, non-limiting mention may be made of titanium dioxide, optionally with surface treatment, zirconium oxide, cerium oxide, zinc oxide, iron oxide (black, yellow brown or red), chromium oxide, manganese violet, ultramarine blue, chromium hydrate and ferric blue. Among organic pigments, non-limiting mention may be made of carbon black, organic lake pigments of barium, strontium, calcium and aluminium, including those certified by the U.S. Food and Drug Administration (FDA) such as D&C and FD&C, and those exempt from FDA certification, such as lakes based on cochineal carmine.

The nacres and nacreous pigments may be chosen from white nacreous pigments such as titanium-covered mica or bismuth oxychloride; coloured nacreous pigments such as titanium mica with iron oxides, titanium mica with, for example, ferric blue and chromium oxide, titanium mica with an organic pigment of the aforementioned type, and also nacreous pigments based on bismuth oxychloride. Use may therefore be made of pigments having goniochromatic properties and metal effect pigments as described in the application filed under number FR 0209246, the content of which is hereby incorporated into the present application.

For example, the colorants may be present in the first and/or second composition in an amount ranging from 0.001 to 60%, such as from 0.01 to 50%, for instance, from 0.1 to 40% by weight, relative to the total weight of the first and/or second composition.

The colorant or filler may additionally be present in the form of a “particle paste”.

Additives and Pharmaceuticals of the First or Second Composition

Either the first or second composition of the present disclosure may further comprise at least one cosmetic or dermatological additive, such as those conventionally employed.

Among cosmetic, dermatological, hygiene and pharmaceutical ingredients that can be used in the composition of the present disclosure, non-limiting mention may be made of moisturizers, vitamins, essential fatty acids, sphingolipids and sunscreens. These additives are used in an amount which is customary for the person skilled in the art, for example, in an amount ranging from 0 to 20%, such as from 0.001 to 15% by weight, relative to the total weight of the composition.

The composition may further comprise any other additive commonly used in such compositions, such as water, antioxidants, perfumes, preservatives and essential oils.

The person skilled in the art will of course take care to select these optional complementary compounds, and/or their amount, such that the advantageous properties of the composition according to the present disclosure are not, or not substantially, adversely affected by the intended addition.

In one aspect of the present disclosure, the compositions as disclosed herein may be prepared in a customary manner by the person skilled in the art. They may be present in the form of a cast product, for example in the form of a stick or crayon, or in the form of a dish, which can be used by direct contact or with a sponge. For instance, the compositions may be in the form of a cast foundation, cast blusher, cast eyeshadow, lipstick, a care base or care balm for the lips, and a concealer product. The compositions may also be in the form of a soft paste or gel, a more or less fluid cream, or a liquid, which may be packaged in a tube. The compositions may therefore constitute foundations, lipsticks, sun products, and skin coloring products.

In one aspect of the present disclosure, the compositions as disclosed herein may be anhydrous and thus comprise less than 5% of water by weight, relative to the total weight of the compositions.

These compositions for topical application may for instance, further comprise a cosmetic, dermatological, hygiene or pharmaceutical composition for the protection, treatment or care of the face, neck, hands and body, for example care cream, sun oil, body gel; a makeup composition for example a makeup gel, cream or stick, an artificial tanning composition, and a skin protection composition.

The compositions according to the present disclosure may be in the form of a dermatological or care composition for the skin and/or epidermal derivatives, or in the form of a sun protection composition or a body hygiene composition, for instance, in the form of a deodorant. In the case of a deodorant form, for example, it may be in an uncolored form. The compositions may therefore be used as a care base for the skin, epidermal derivatives or lips, such as lip balms, protecting the lips from cold and/or sun and/or wind, or care cream for the skin, nails or hair.

The compositions according to the present disclosure are for example, lipsticks in the form of a stick or in fluid form.

The compositions of the present disclosure must of course be cosmetically and dermatologically acceptable; that is, the compositions must comprise a non-toxic physiologically acceptable medium that can be applied to the skin, epidermal derivatives or lips of the face of human beings. A cosmetically acceptable composition for the purposes of the present disclosure is a composition whose appearance, odor and feel are pleasant.

Each composition of the two-coat makeup product according to the present disclosure may be present in any form that is normally used for topical application, for example, in the form of an oily or aqueous solution, an oily or aqueous gel, an oil-in-water or water-in-oil emulsion, a multiple emulsion, a dispersion of oil in water which is mediated by vesicles, the vesicles being situated at the oil/water interface, or a powder. Each composition may be fluid or solid.

For example, the first or the second composition, or both, may have a continuous fatty phase and for instance, may be in anhydrous form, and may contain less than 5% of water, such as than 1% of water, relative to the total weight of the first or second composition. In one aspect of the present disclosure, the overall two-coat makeup product is in anhydrous form.

Each first and second composition may have the appearance of a lotion, cream, ointment, soft paste, salve, a cast or moulded solid, for example, in stick form or dish form, or else a compacted solid.

In another aspect of the present invention, each composition is in the form of a more or less rigid stick.

Each composition may be packaged separately in a single container, for example in a twin-compartment pen, the base composition being delivered by one end of the pen and the top composition being delivered by the other end of the pen, each end being closed, for instance, tightly, by means of a cap.

The composition that is applied in a first coat may be, for example, in solid form, allowing more practical application, better temporal stability and temperature stability of the composition, and a precise line of the makeup, which is highly desirable in the case of a lipstick or an eyeliner.

The product according to the present disclosure may be used for example, for making up the skin and/or lips and/or epidermal derivatives, in accordance with the nature of the ingredients employed. For example, the product as disclosed herein may be in the form of a solid foundation, a lipstick or lipstick paste, a concealer product, or an eye contour product, an eyeliner, a mascara, an eye shadow, a body makeup product or else a skin colouring product.

The product may be for example, a lipstick.

For further example, the first and/or the second composition may be in solid form.

The topcoat has, for instance, properties of care, gloss and transparency.

The disclosure further provides a lip product, a foundation, a tattoo, a blusher or an eyeshadow, which comprises first and second compositions as described above.

The compositions as disclosed herein may be obtained by heating the various constituents to the melting temperature of the highest waxes, then casting the melted mixture in a mould, such as a dish or glove finger. They may also be obtained by extrusion as described in application EP-A-0 667 146.

When the physiologically acceptable medium of the first and/or second composition comprises a liquid organic phase, this medium may for example, comprise water dispersed or emulsified in the said liquid organic phase.

In one aspect of the present disclosure, the compositions as disclosed herein may be prepared in customary manner by the person skilled in the art. They may be in the form of a cast product and, for example, in the form of a stick or wand, in the form of a soft paste in a pot, or in the form of a dish which can be used by direct contact or with a sponge. For example, the compositions constitute, together or separately, a cast foundation, blusher or cast eyeshadow, coloured for example, lipstick, lip gloss or concealer. They may also each be present in the form of a soft paste or else a gel or a more or less fluid cream. In that case they may constitute fluid or pastelike foundations or lipsticks, lip glosses, sun products or skin colouring products, eyeliners or body makeup products or else may exhibit care properties, in which case they may be in the form of a lipcare base or balm.

Each composition of the product according to the present disclosure may be present in any form normally used for topical application, for example in the form of an oily or aqueous solution, an oily or aqueous gel, an oil-in-water or water-in-oil emulsion, a multiple emulsion, a dispersion of oil in water mediated by vesicles, the vesicles being situated at the oil/water interface, or a powder. Each composition may be fluid or solid.

In one aspect of the present disclosure, the first or the second composition, or both, have a continuous fatty phase and be, for example, in anhydrous form, and thus may comprise less than 5% of water by weight, for instance, less than 1% of water, relative to the total weight of the first or second composition. For example, the overall two-coat makeup product can be in anhydrous form.

Each first and second composition may have the appearance of a lotion, cream, ointment, soft paste, salve, a cast or molded solid, such as in stick form or dish form, or else a compacted solid.

Each composition may be, for example, in the form of a more or less rigid stick.

The at least one first and at least one second compositions can be packaged in separate containers, or in discrete compartments of a single container.

Each composition may be packaged separately in a single container, for example in a twin-compartment pen, the base composition being delivered by one end of the pen and the top composition being delivered by the other end of the pen, each end being closed, for instance, tightly, by means of a cap.

In one aspect of the present disclosure, the composition that is applied in a first coat is in solid form, allowing more practical application, better temporal and temperature stability of the composition, and a precise line of the makeup, which is highly desirable in the case of a lipstick or an eyeliner.

The invention is illustrated in more detail in the following examples. The percentages are percentages by weight.

### EXAMPLE 1

### Polymer Dispersion

A dispersion was prepared of an uncrosslinked copolymer of methyl acrylate and acrylic acid in a 95/5 ratio in isododecane by the method of Example 1 of EP-A-749 746, replacing the heptane with isododecane. This gave a dispersion of poly(methyl acrylate/acrylic acid) particles surface-stabilized in isododecane by a block polystyrene/copoly(ethylene-propylene) diblock copolymer sold under the name Kraton G1701, having a solids content of 25% by weight.

### EXAMPLE 2

### Lipstick

| | |
|---|---|
| Polyethylene wax | 10.5% |
| (weight-average molecular mass: 500) | |
| Linear fatty alcohols | 2.5% |
| (Performacol 550 alcohol, sold by | |
| New Phase Technologies) | |
| Dispersion of Example 1 | 68% |
| Sucrose acetate isobutyrate | 5% |
| (Eastman SAIB sold by Eastman Chemical) | |
| Pigment paste | 13.5% |
| Fragrance | 0.5% |

A heating vessel was charged with the polyethylene wax, the C30-C₅₋₀ alcohols and the pigment paste, which were heated at 100° C. with magnetic stirring to give a homogeneous mixture. The composition of the pigment paste was 70% pigments, 1% poly(12-hydroxystearic) stearate and 29% hydrogenated polyisobutene. Subsequently, the dispersion of Example 1 and the sucrose acetate isobutyrate were added, while maintaining the temperature and the stirring until the mixture homogenized. The composition was cast into molds. The resulting sticks were homogeneous in colour and went on well. They resulted in a deposit on the lips which had good staying power, did not migrate or transfer and was not sticky.

### EXAMPLE 3

### Lipstick as First Composition

| | |
|---|---|
| Polystyrene/poly(ethylene-propylene) | 0.5% |
| Kraton G-1650E | |
| (sold by Kraton) | |
| Dispersion of Example 1 | 65% |
| Polycaprolactone | 10% |
| (Capa 2125, manufactured by Solvay) | |
| Sucrose acetate isobutyrate | 10% |
| (Eastman SAIB, sold by Eastman Chemical) | |
| Pigment paste | 13.5% |
| Fragrance | qs |
| Preservative | qs |

The polycaprolactone was melted at 100° C. and then the pigment paste, whose composition was identical to that of Example 2, was added with stirring. Subsequently the other ingredients were added until a homogeneous mixture was obtained. The mixture was subsequently cooled to ambient temperature and packaged in pots.

### EXAMPLE 4

### Liquid Second Composition

| | |
|---|---|
| Polydimethylsiloxane sold | 40% |
| as Silbione 70047 V by Rhodia | |
| (500 000 cSt - 250 000 g/mol) | |
| Polydimethylsiloxane sold | 60% |
| by Dow Coming as DC200 (5 cSt) | |

The two ingredients were mixed at 70° C. using a Rayneri stirrer.

The composition according to the present disclosure had a viscosity of approximately 8±1 Pa.s, the viscosity was measured at 25° C. with a Rheomat RM 180 instrument, using spindle 4 rotating at 200 rpm, after 10 minutes in accordance with measurement system 75.

### EXAMPLE 5

### Solid Second Composition

| | % by weight |
|---|---|
| Silicone oil (PDMS) | 25 |
| DC200 from Dow Corning (5 cST) | |
| DIMETHICONE (and) DIMETHICONOL | 61 |
| D2-9085 from Dow Corning (1550 cSt) | |
| TRIFLUOROPROPYL DIMETHICONE (100 Cst) | 1 |
| X22-819 from Shin Etsu | |
| C30-45 ALKYL DIMETHICONE | |

The silicone oil, dimethiconol and fluorinated dimethicone were mixed with heating until a homogeneous mixture was formed. The C₃₀-C₄₅ alkyl dimethicone was then added to the above mixture, which had been heated to 110° C. The polyethylene wax was subsequently added gradually until a homogeneous mixture was obtained. The mixture was cooled to 90-95° C. and then poured into molds, which were cooled at −20° C. for thirty minutes. The resulting sticks were subsequently demolded.

What is claimed is:

1. A cosmetic product comprising at least two compositions wherein:
a first composition comprises a liquid fatty phase and polymer particles dispersed in the liquid fatty phase, and
a second composition comprises at least one high molecular weight polymer having a weight-average molecular mass of greater than or equal to 200,000 g/mol.

2. The cosmetic product according to claim 1, wherein the at least one high molecular weight polymer is a silicone polymer.

3. The cosmetic product according to claim 1, wherein the at least one high molecular weight polymer has a weight-average molecular mass ranging from 200,000 g/mol to 4,000,000 g/mol.

4. The cosmetic product according to claim 3, wherein the at least one high molecular weight polymer has a weight-average molecular mass ranging from 200,000 g/mol to 2,500,000 g/mol.

5. The cosmetic product according to claim 4, wherein the at least one high molecular weight polymer has a weight-average molecular mass ranging from 200,000 g/mol to 2,000,000 g/mol.

6. The cosmetic product according to claim 1, wherein the at least one high molecular weight polymer is chosen from dimethiconols, fluorosilicones, and dimethicones.

7. The cosmetic product according to claim 1, wherein the at least one high molecular weight polymer corresponds to the formula (IV): 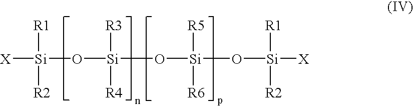
wherein:
R₁, R₂, R₅ and R₆, which may be identical or different, are each chosen from alkyl radicals having from 1 to 6 carbon atoms that may be optionally substituted by at least one fluorine atom,
R₃ and R₄, which may be identical or different, are each chosen from aryl radicals and from alkyl radicals having from 1 to 6 carbon atoms,
X is chosen from alkyl radicals having from 1 to 6 carbon atoms, a hydroxyl radical, vinyl radicals, allyl radicals, and alkoxy radicals having from 1 to 6 carbon atoms,
n and p are chosen such that the at least one high molecular weight polymer has a weight-average molecular mass of greater than or equal to 200,000 g/mol.

8. The cosmetic product according to claim 7, wherein the at least one high molecular weight polymer is a dimethiconol of formula (IV) wherein R₁ to R₆ are all methyl groups, X is a hydroxyl group, p=0 and n ranges from 2,000 to 40,000.

9. The cosmetic product according to claim 8, wherein n ranges from 3,000 to 30,000.

10. The cosmetic product according to claim 8, wherein the at least one high molecular weight polymer has a weight-average molecular mass ranging from 1,500,000 to 2,000,000 g/mol.

11. The cosmetic product according to claim 1, wherein the at least one high molecular weight polymer has a viscosity ranging from 1,000 to 10,000,000 cSt, measured in accordance with standard ASTM D-445.

12. The cosmetic product according to claim 2, wherein the at least one high molecular weight silicone polymer is introduced into the second composition in the form of a mixture with at least one liquid silicone compound, wherein the viscosity of the at least one liquid silicone compound ranges from 0.5 to 10,000 cSt, measured in accordance with standard ASTM D-445.

13. The cosmetic product according to claim 12, wherein the viscosity of the at least one liquid silicone compound ranges from 0.5 to 500 cSt, measured in accordance with standard ASTM D-445.

14. The cosmetic product according to claim 13, wherein the at least one liquid silicone compound ranges from 1 to 10 cSt, measured in accordance with standard ASTM D-445.

15. The cosmetic product according to claim 12, wherein the ratio of the at least one high molecular weight silicone polymer to the at least one liquid silicone compound in the mixture ranges from 10/90 to 20/80.

16. The cosmetic product according to claim 12, wherein the viscosity of the mixture of the at least one high molecular weight polymer of formula (IV) and of the at least one liquid silicone compound ranges from 1,000 to 10,000 cSt, measured in accordance with standard ASTM D-445.

17. The cosmetic product according to claim 7, wherein the at least one high molecular weight polymer is a dimethicone of formula (IV) wherein R₁ to R₆ and X are all methyl groups.

18. The cosmetic product according to claim 15, wherein the at least one high molecular weight polymer is chosen from polydimethylsiloxanes with a weight-average molecular weight ranging from 200,000 g/mol to 300,000 g/mol.

19. The cosmetic product according to claim 18, wherein the at least one high molecular weight polymer is chosen from polydimethylsiloxanes with a weight-average molecular weight ranging from 240,000 g/mol to 260,000 g/mol.

20. The cosmetic product according to claim 2, wherein the at least one high molecular weight silicone polymer is chosen from fluorosilicones.

21. The cosmetic product according to claim 1, wherein the polymer particles of the first composition have an average size ranging from 5 to 800 nm.

22. The cosmetic product according to claim 1, wherein the polymer particles of the first composition are insoluble in water-soluble alcohols.

23. The cosmetic product according to claim 1, wherein the polymer particles of the first composition are chosen from at least one of polyurethanes, polyurethane-acrylics, polyureas, polyurea-polyurethanes, polyester-polyurethanes, polyether-polyurethanes, polyesters, polyester amides, fatty-chain polyesters, alkyds; acrylic and/or vinyl polymers and/or copolymers; acrylic-silicone copolymers; polyacrylamides; silicone polymers, and fluoro polymers.

24. The cosmetic product according to claim 1, wherein the polymer particles of the first composition are capable of forming a film.

25. The cosmetic product according to claim 1, wherein the polymer particles are present in the first composition in an amount ranging from 2 to 40% by dry weight of the particles, relative to the total weight of the first composition.

26. The cosmetic product according to claim 25, wherein the polymer particles are present in the first composition in an amount ranging from 5 to 30% by dry weight of the particles, relative to the total weight of the first composition.

27. The cosmetic product according to claim 26, wherein the polymer particles are present in the first composition in an amount ranging from 8 to 20% by dry weight of the particles, relative to the total weight of the first composition.

28. The cosmetic product according to claim 1, wherein the polymer particles of the first composition are surface-stabilized by a stabilizer.

29. The cosmetic product according to claim 28, wherein the stabilizer is chosen from block polymers and graft block polymer, including at least one block resulting from the polymerization of a diene and at least one block of a vinyl polymer.

30. The cosmetic product according to claim 29, wherein the stabilizer is a diblock polymer.

31. The cosmetic product according to claim 1, wherein the liquid fatty phase of the first composition comprises at least one volatile oil.

32. The cosmetic product according to claim 31, wherein the at least one volatile oil is present in the first composition in an amount ranging from 20 to 90% by weight, relative to the total weight of the first composition.

33. The cosmetic product according to claim 32, wherein the at least one volatile oil is present in the first composition in an amount ranging from 30 to 80% by weight, relative to the total weight of the first composition.

34. The cosmetic product according to claim 33, wherein the at least one volatile oil is present in the first composition in an amount ranging from 40 to 70% by weight, relative to the total weight of the first composition.

35. The cosmetic product according to claim 31, wherein the at least one volatile oil is chosen from C₈-C₁₆ isoalkanes.

36. The cosmetic product according to claim 35, wherein the at least one volatile oil is chosen from isododecane and isohexadecane.

37. The cosmetic product according to claim 1, wherein the liquid fatty phase of the first composition is an apolar non-volatile liquid fatty phase.

38. The cosmetic product according to claim 37, wherein the apolar non-volatile liquid phase comprises hydrogenated polyisobutene.

39. The cosmetic product according to claim 1, wherein the first composition comprises a colloidal dispersion of colored particles.

40. The cosmetic product according to claim 39, wherein the colored particles are chosen from at least one of pigments and nacres.

41. The cosmetic product according to claim 39, wherein the colloidal dispersion is present in the first composition in an amount ranging from 0.5% to 60% by weight, relative to the total weight of the first composition.

42. The cosmetic product according to claim 41, wherein the colloidal dispersion is present in the first composition in an amount ranging from 2% to 40% by weight, relative to the total weight of the first composition.

43. The cosmetic product according to claim 42, wherein the colloidal dispersion is present in the first composition in an amount ranging from 2% to 30% by weight, relative to the total weight of the first composition.

44. The cosmetic product according to claim 39, wherein the particles of the colloidal dispersion are stabilized with a dispersant.

45. The cosmetic product according to claim 1, wherein the first composition further comprises at least one gelling agent which gels the liquid fatty phase and which is chosen from polymeric gelling agents.

46. The cosmetic product according to claim 45, wherein the at least one gelling agent is present in the first composition in an amount ranging from 0.05% to 5% by weight, relative to the total weight of the first composition.

47. The cosmetic product according to claim 46, wherein the at least one gelling agent is present in the first composition in an amount ranging from 0.1% to 3% by weight, relative to the total weight of the first composition.

48. The cosmetic product according to claim 47, wherein the at least one gelling agent is present in the first composition in an amount ranging from 0.2% to 2% by weight, relative to the total weight of the first composition.

49. The cosmetic product according to claim 45, wherein the at least one gelling agent is chosen from polycaprolactones.

50. The cosmetic product according to claim 45, wherein the at least one gelling agent is chosen from amorphous block copolymers of styrene and of olefin.

51. The cosmetic product according to claim 1, wherein the first composition comprises at least one linear hydrocarbon wax.

52. The cosmetic product according to claim 51, wherein the at least one linear hydrocarbon wax is present in the first composition in an amount ranging from 2% to 30% by weight, relative to the total weight of the first composition.

53. The cosmetic product according to claim 52, wherein the at least one linear hydrocarbon wax is present in the first composition in an amount ranging from 5% to 20% by weight, relative to the total weight of the first composition.

54. The cosmetic product according to claim 53, wherein the at least one linear hydrocarbon wax is present in the first composition in an amount ranging from 5% to 15% by weight, relative to the total weight of the first composition.

55. The cosmetic product according to claim 1, wherein at least one of the at least two compositions is in anhydrous form.

56. The cosmetic product according to claim 1, wherein it is in a form chosen from a foundation, a blusher, an eyeshadow, a lipstick, a product having care properties, an eyeliner, a concealer, and a body makeup product.

57. The cosmetic product according to claim 56, wherein it is in the form of a lipstick.

58. The cosmetic product according to claim 1, wherein at least one of the at least two compositions is in solid form.

59. A method of making up the skin and/or lips and/or epidermal derivatives, comprising
applying to the skin, lips and/or epidermal derivatives a first coat of a first composition comprising a liquid fatty phase and polymer particles dispersed in the liquid fatty phase, and then applying over all or part of the first coat, a second coat of a second composition comprising at least one high molecular weight polymer having a weight-average molecular mass of greater than or equal to 200,000 g/mol.

60. A makeup kit comprising a cosmetic product comprising at least two compositions wherein,
a first composition comprises a liquid fatty phase and polymer particles dispersed in the liquid fatty phase, and
a second composition comprises at least one high molecular weight polymer having a weight-average molecular mass of greater than or equal to 200,000 g/mol.

61. The makeup kit according to claim 60, wherein the at least two compositions are packaged in separate containers.

62. The makeup kit according to claim 59, wherein the at least two compositions are packaged in discrete compartments of a single container.

63. A method for providing the skin and/or lips and/or epidermal derivatives with a makeup that exhibits at least one characteristic chosen from comfort, gloss, non-transfer properties, non-migration properties, and staying power, said method comprising:
mixing a first composition comprising a liquid fatty phase and polymer particles dispersed in the liquid fatty phase with
a second composition comprising at least one high molecular weight polymer having a weight-average molecular mass of greater than or equal to 200 000 g/mol,
to make a cosmetic makeup product and then
applying said cosmetic makeup product to the skin and/or lips and/or epidermal derivatives.

64. A made-up substrate comprising a first coat of a first composition comprising a fatty phase and polymer particles dispersed in the fatty phase and a second coat of a second composition deposited over all or part of the first coat and comprising a high molecular weight polymer having a weight-average molecular mass of greater than or equal to 200 000 g/mol.
